(19) United States
(12) Patent Application Publication
(10) Pub. No.: US 2010/0137186 A1
(43) Pub. Date: Jun. 3, 2010

(54) USE OF AN ACTIVE PRINCIPLE ORIGINATING FROM AMARANTH (AMARANTHUS) FOR PREPARING A COMPOSITION INTENDED TO ACTIVATE CELLULAR ENERGY AND TO PROTECT THE SKIN FROM OXIDATIVE DAMAGE

(75) Inventors: Claude Dal Farra, Opio (FR); Nouha Domloge, Valbonne (FR); Jean-Marie Botto, Valbonne (FR)

Correspondence Address: YOUNG & THOMPSON, 209 Madison Street, Suite 500, Alexandria, VA, 22314, US

(73) Assignee: Societe D'Extraction Des Principes Actifs S.A. (ISP Vincience)

(21) Appl. No.: 12/597,867

(22) PCT Filed: Apr. 23, 2008
(86) PCT No.: PCT/FR2008/000576

§ 371 Date: Oct. 27, 2009

(30) Foreign Application Priority Data
Apr. 27, 2007 (FR) 0703062

(51) Int. Cl. A61K 8/64; C07K 2/00; A61Q 19/00; A61Q 19/08

(52) U.S. Cl. 514/2; 530/300

(57) ABSTRACT

Methods of administering in a cosmetic composition or a pharmaceutical composition, an effective amount of a peptide active principle originating from amaranth of the species Amaranthus hypochondriacus. The active principle is intended to activate cellular energy and to protect the skin from oxidativ damage, and the active principle may be used alone or in association with at least one other active principles. The active principle may also be used in a cosmetic treatment for protecting the skin and the appendages from external aggressions and to combat cutaneous aging.

The present invention is in the cosmetic and pharmaceutical domain, and more particularly in the domain of dermatology. The present invention concerns the use in a cosmetic composition or for preparing a pharmaceutical composition, of an effective quantity of a peptide active principle originating from amaranth of the species Amaranthus hypochondriacus, said active principle, or a composition containing it, being intended to activate cellular energy and to protect the skin from oxidative damage. The active principle may be used alone or in association with at least one other active principle. The invention also concerns a cosmetic-treatment procedure intended to protect the skin and the appendages from external aggressions and to combat cutaneous aging. The said active principle can also be used to prepare pharmaceutical compositions intended to prevent or combat pathologies linked to oxidation processes or even certain pathologies of aging.

The term “appendages” according to the invention encompasses the assemblage of keratinic appendices exhibited on the body surface, in particular the hair, eyelashes, eyebrows, nails, and hair.

The skin is a vital organ that covers the entire surface of the body and provides protective, sensitive, immune, metabolic, or even thermoregulatory functions. The skin, like other organs, is subject to aging. So, one of the major mechanisms implicated in the processes of aging is the accumulation of oxidative damage in essential molecules such as membrane lipids, proteins, DNA, and most particularly mitochondrial DNA (DNAmt).

Oxidative damage is caused by free radicals, chemically unstable and very reactive species generated by intracellular metabolism or external aggressions. Among these external aggressions, UV rays, toxins, atmospheric pollutants, and alimentary oxidants may be cited. Premature aging is observed in the skin, occurring in areas exposed to radiation, characterized by phenomena of alterations in the macromolecules (lipid peroxidation, carbonylation of proteins) affecting, in particular, elastin, collagen, and fibronectin. Progressive decline with age can also be shown in the mitochondrial functions, probably linked to the accumulation of mutations on DNAmt (K. Singh (2004), Ann. NY Acad. Sci., 1019).

One of the important consequences of the accumulation of oxidative damage is the reduction in the capacity of the cell to produce ATP (Porteous et al. (1998), Eur. J. Biochem. 257(1), 192-201). Thus, the phenomenon of cellular aging is in proportion to the oxidative damage the cell undergoes, as well as to the process of producing the energy the cell needs to survive.

The body possesses defense mechanisms capable of trapping or of transforming free radicals (enzymes, glutathione, vitamins A and E, coenzyme Q10, etc.). However, these antioxidant defense systems often prove to be insufficient under the numerous stresses and external aggressions to which the body, and the skin in particular, are subjected.

In this context, the particular properties of coenzyme Q10 appear to be particularly interesting.

Coenzyme Q10 (or ubiquinone) is a coenzyme present in the mitochondrial complexes implicated in oxidative phosphorylation leading to the production of ATP (Mitchell et al. (1976), Mitchell et al. (1990)). The other fundamental property of coenzyme Q10 is as an antioxidant, neutralizing free radicals (Beyer et al. (1990), Vittalba et al. (1997)).

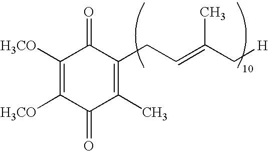

Coenzyme Q10 is a benzoquinone derivative flanked by a long isoprene side chain, most often composed of ten isoprenoid units (whence the name coenzyme Q10). As this coenzyme is not soluble in water, it is only encountered in lipid membranes like the internal membrane of the mitochondrion, where it can freely diffuse among the membrane phospholipids.

Coenzyme Q10 can exist in three oxidation states: a reduced form (CoQH2 or UQH2), an oxidized state (CoQ10), and an intermediate form, the radical ubisemiquinone)(Q⁰).

The biosynthesis of coenzyme Q10 starts with thyrosine for the quinone core and with farnesyl pyrophosphate for the side chain. The enzyme responsible for this last reaction, which is an essential step in the biosynthesis of coenzyme Q10, is transphenyl transferase (or polyphenyl transferase).

The search for compounds capable of stimulating the synthesis of coenzyme Q10 or the energy synthesis of ATP, and/or protecting the cells from damage caused by free radicals is an important concern of medical research and of cosmetics. Solutions have thus been proposed such as the intake of substances of peptide origin exhibiting antioxidant properties (WO2005097060, JP2006131626) or vitamin C (US 2004/0086526) or L-ergothionine (WO 9836748).

The present invention has as its principal objective the use of a peptide active principle originating from the hydrolysis of amaranth of the species Amaranthus hypochondriacus, capable of protecting the skin from external aggressions and of combating cutaneous aging. The said active principle could be used alone or in association with at least one other active principle. The inventors have highlighted a therapeutic activity, notably a dermatological and cosmetic one, of such an active principle. It has been particularly brought out that this active principle, when applied on the skin, has a strong protective action against oxidative damage to which the skin is subjected and promotes the synthesis of ATP in a significant way, as well as the synthesis or the activity of the enzyme transphenyl transferase and of coenzyme Q10. This new active principle, capable of increasing cellular energy and of protecting the skin from oxidative damage thus opens up new therapeutic and cosmetic perspectives.

An “active principle capable of increasing cellular energy and of protecting the skin from oxidative damage” is understood to be any substance of vegetable origin, and more particularly originating from amaranth of the species Amaranthus hypochondriacus capable of increasing the synthesis of intracellular ATP and of exhibiting protective properties in cells or tissues subjected to an oxidative stress of physico-chemical or environmental origin. The active principle capable of increasing cellular energy and of protecting the skin from oxidative damage is, according to the invention, an extract of peptide nature and originates from the hydrolysis of proteins of amaranth of the species Amaranthus hypochondriacus. “Of peptide nature” is understood to be a mixture of compounds represented by peptides of polypeptides.

The term “peptide” designates a chaining of two or several amino acids linked to one another by peptide bonds or by modified peptide bonds, the term “polypeptide” designating a peptide of greater size.

The expression “biologically active” is understood to be “that posses an in vivo or in vitro activity characteristic of the activity of the active principle according to the invention”.

The term “hydrolysate or originating from the hydrolysis” designates any substance or mixture of substances, or isolated preparation, obtained after hydrolysis of vegetable matter.

The peptide active principle according to the invention is obtained by the extraction of proteins of vegetable origin, after a controlled hydrolysis that releases biologically active peptide fragments.

A great many proteins found in plants are likely to contain these sequences within their structure. Controlled hydrolysis enables these peptide fragments to be released. It is possible, but not necessary to achieve the invention, either to extract the proteins concerned first and then to hydrolyze them, or to perform the hydrolysis first on a raw extract and to subsequently purify the peptide fragments. It is also possible to use certain hydrolyzed extracts without purifying the peptide fragments in them which correspond to peptides with the general formula (I) according to the invention, but at the same time ensuring the presence of the said fragments by appropriate analytical means.

In order to achieve the extraction, the entire plant may be used or a specific part of the plant (leaf, berry, etc.).

The amaranth is an annual plant in the family Amaranthaceae belonging to the genus Amaranthus, some species of which are cultivated as ornamental plants for their flowers with a spectacular ear, and sometimes as culinary plants, for their edible leaves, like spinach greens or for their seeds.

According to the invention, the species used is Amaranthus hypochondriacus, the vegetable material used is the seed, and preferentially the seed separated from its husk by a dehusking step.

In a first step, the plant is crushed using a plant crusher. The powder thus obtained may ultimately be “delipidated” with the aid of a traditional organic solvent (like, for example, alcohol, hexane, or acetone).

Then the extraction of proteins from the plane is performed according to the modified traditional procedure (Osborne (1924); the plant crush is placed in suspension in an alkaline solution containing an adsorbent product of the insoluble polyvinyl polypyrrolidone (PPVP) type (0.01-20%). Indeed, it has been observed that only operations of hydrolysis and final purification are facilitated by this means. The concentration of substances of the phenolic type which interact with the proteins are thus found to be reduced.

The soluble fraction is collected after the centrifuging and filtering steps, this raw solution then constituting as first form of the extract, containing the proteins, the glucides, and possibly lipids.

The proteins are then precipitated, varying the ionic strength when acidifying the medium, which enables the soluble components and the nucleic acids to be eliminated.

The precipitate is then washed with an organic solvent such as, for example, ethanol or methanol, and the solvent is evaporated by drying in a vacuum. The precipitate, rich is proteins, is placed in solution again in water or another solvent and then consists of a more purified form of the hydrolysate.

The extraction may also be performed in a neutral or acidic medium, always in the presence of polyvinyl polypyrrolidone. After a filtration step, the precipitation step is them performed using a traditional precipitation agent such as the salts (sodium chloride, ammonium sulfate) or an organic solvent (alcohol, acetone). The precipitate obtained may be separated from the agents of precipitation by dialysis after being placed again in solution in water or another solvent.

The proteinic fraction isolated according to the invention is then hydrolyzed under controlled conditions to generate soluble polypeptides and peptides. Hydrolysis is defined as a chemical reaction implying the cleaving of a molecule by water, this reaction being able to be performed in a neutral acidic, or basic medium. According to the invention, the hydrolysis is achieved by chemical means and/or in an advantageous manner by proteolytic enzymes. The use of endoproteases of vegetable origin may then be cited (papayine, bromelaine, ficine) and of micro-organisms (Aspergillus, Rhizopus, Bacillus, etc.).

For the same reasons as before, during this step of controlled hydrolysis, a quantity of polyvinyl pyrrolidone is added to the reaction medium. After filtering, the solution obtained constitutes the active hydrolysate. The active hydrolysate may then be purified in order to select the molecular weights and the nature of the peptides generated. Fractionation may be performed to advantage by ultrafiltration and/or by a chromatographic-type method.

Any one of the more or less purified forms of the hydrolysate is then solubilized in water or in any mixture containing water, and then sterilized by ultrafiltration.

The vegetable hydrolysate obtained according to the invention is analyzed qualitatively and quantitatively for its physico-chemical characteristics and its content in compounds of a protein and peptide nature. Compounds of a peptide nature are understood to be the protein fragments, peptides, and free amino acids present in the mixture. The peptides, amino acids, and protein fragments are measured according to traditional techniques, well know to the professional.

Thus, according to an advantageous embodiment of the invention, the active vegetable hydrolysate has a pH between 4 and 7, and preferentially between 5 and 6, a dry extract titering between 1 and 8 g/l, and preferably between 2 and 5 g/l. Its content in compounds of a peptide nature is between 0.12 and 5 g/l, and preferentially between 0.5 and 2 g/l, and its content in sugars is from 0.5 to 2.5 g/1.

According to one advantageous embodiment of the invention, the active principle according to the invention is solubilized in advance in one or several solvents traditionally used by the professional, such as water, glycerol, ethanol, propylene glycol, butylene glycol, dipropylene glycol, ethoxylated or propoxylated diglycols, cyclic polyols, Vaseline, a vegetable oil, or any mixture of these solvents.

According to yet another advantageous embodiment of the invention, the active principle according to the invention is solubilized in advance in a cosmetic or pharmaceutical vehicle like the liposomes or adsorbed onto powdered organic polymers or mineral supports like the talcs and bentonites, and is more generally solubilized in, or fixed upon, any cosmetically or pharmaceutically acceptable vehicle.

The compositions according to the invention could be applied in any appropriate way, particularly oral, parenteral, or externally topical, and their formulation will be adapted by the professional, in particular for cosmetic or dermatological compositions. Advantageously, the compositions according to the invention are intended for administration by topical, cutaneous means. These compositions shall therefore contain a cosmetically and/or dermatologically acceptable medium, that is, compatible with the skin and the appendages, and they cover all the cosmetic or dermatological forms. These compositions could, in particular, be in the form of creams, oil-in-water or water-in-oil emulsions or multiple emulsions, solutions, suspensions, gels, milks, lotions, sticks, or even powders, adapted to an application onto the skin, the lips, and/or the appendages.

These compositions include the excipients necessary for their formulation, such as solvents, thickeners, diluents, surfactants, antioxidants, colorants, preservatives, or perfumes.

Of course, the professional will take care to choose possible supplemental compounds, active or not, and/or their quantity, such that the advantageous properties of the mixture are not altered by the addition envisioned.

The composition usable according to the invention can, in particular, consist of a composition for hair care, and particularly a shampoo, a conditioner, a blow-dry lotion, a treatment lotion, a cream or a styling gel, a restructuring lotion for the hair, a mask, etc. The cosmetic composition according to the invention can be used particularly in treatments implementing an application that is followed or not by a rinse, or even in the form of shampoo.

It can also come in the form of a dye or a mascara to be applied with brush or comb, in particular on the eyelashes, eyebrows, or hair.

Advantageously, the usable compositions contain, in addition, at least one other active agent promoting the action of the peptide active principle according to the invention. Thus, the composition according to the invention may associate, with the active principle, capable of increasing cellular energy and of protecting the skin from oxidative damage, active agents having an antioxidant action, or else stimulating the synthesis of dermal macromolecules, or else stimulating energy metabolism. For example, as an active principle having an anti-radical or antioxidant action, vitamin C, vitamin E, or coenzyme Q10, or the polyphenolic extracts of plants may be cited. Active anti-radical principles are understood to be any compound capable of trapping free radicals. These active principles are capable of blocking the chain reactions of free radicals before the final stages of degradation of the biological constituents of the skin, and because of this they have an antioxidant activity.

As an active agent stimulating the synthesis of dermal macromolecules (laminin, fibronectin, collagen), the collagen peptide marketed under the name of Collaxyl® by the Vincience Company may be cited.

Finally, the active principle marketed under the name of GP4G® by the Vincience Company may be cited as an active agent stimulating energy metabolism.

From another angle, the composition according to the invention may be a sun-related composition, that is, a composition that aids in protection against solar radiation. Thus, there may be advantageously added to the composition according to the invention active agents aiding in solar protection such as, for example, solar filters.

It is quite obvious that the invention is directed toward mammals in general and more particularly toward human beings.

The effective quantity of active principle corresponds to the quantity of amaranth hydrolysate obtained according to the invention necessary to obtain the result sought, that is to say, to increase the synthesis of ATP, to protect the skin from oxidative damage, and more generally to protect the skin from external aggressions and to prevent or treat cutaneous aging.

According to an advantageous embodiment of the invention, the active principle originating from amaranth is present in the compositions of the invention in a concentration between approximately 0.0001 and 20%, and preferentially in a concentration between approximately 0.05 and 5%, relative to the total weight of the final composition.

These compositions could come particularly in the form of an aqueous, hydroalcoholic, or oily solution, an oil-in-water or water-in-oil emulsion, or multiple emulsions. They can also come in the form of creams, suspensions, or even powders, adapted to application onto the skin, the mucous membranes, the lips, and/or the appendages. These compositions can be more or less fluid and have the appearance of a cream, a lotion, a milk, a butter, an ointment, a gel, a paste, or a mousse. They can also come in solid form like a stick or be applied on the skin in the form of an aerosol. They can be used as a care product and/or as a makeup product for the skin.

These compositions include, in addition, any additive commonly used in the application domain envisioned, as well as the adjuvants necessary for their formulation, such as solvents, thickeners, diluents, antioxidants, colorants, solar filters, self-bronzing agents, pigments, vehicles, preservatives, perfumes, odor absorbents, active cosmetic or pharmaceutical agents, essential oils, vitamins, essential fatty acids, surfactants, film-forming polymers, etc.

In any case, the professional will take care that these adjuvants, as well as their proportions, are chosen in such a way as not to harm the advantageous properties sought in the composition according to the invention. These adjuvants can, for instance, correspond to 0.01 to 20% of the total weight of the composition. When the composition of the invention is an emulsion, the fatty phase may represent 5 to 80% by weight and preferably 5 to 50% by weight relative to the total weight of the composition. The emulsifiers and co-emulsifiers used in the composition will be chosen from among those traditionally used in the domain considered. For example, they may be used in a proportion of 0.3 to 30% by weight relative to the total weight of the composition.

By means of its particular activities, the active principle according to the invention could be used advantageously in a cosmetic composition or for the preparation of a pharmaceutical composition.

In particular, the active principle according to the invention could be used advantageously in a cosmetic composition intended to combat in a preventive and/or curative manner the manifestations of cutaneous aging and, more specifically, to combat and/or prevent photo-induced aging (photo-aging). Cutaneous manifestations of aging are understood to be any alteration of the external appearance of the skin due to aging, such as, for example, wrinkles and fine wrinkles, shriveled skin, flabby skin, thin skin, lack of elasticity and/or tonus of the skin, dull skin without a glow or pigmentation spots on the skin, as well as any internal alteration of the skin that is not manifested systematically in an altered external appearance, such as, for example, any internal degradation of the skin following an exposure to ultraviolet (UV) rays. The active principle according to the invention, or the composition containing it, will enable combating, in particular, the loss of elasticity and firmness of the skin.

The use of the peptide, or of a composition containing it, will allow the skin and the appendages to be protected and to better resist environmental stresses. Thus, an essential aspect of the invention is the use of the active principle according to the invention, in a cosmetic composition for protecting the skin and the appendages against oxidative damage, thanks to a protective activity with respect to reactive species of oxygen, the said active principle being used to advantage as an active antioxidant principle, and/or as an active anti-radical principle, and/or as an active antiglycation principle. An active anti-radical principle is understood to be any compound capable of trapping free radicals before the final stages of degradation of the biological constituents of the skin, and they are then called antioxidant compounds. An active antiglycation principle is understood to be any compound capable of limiting the cellular damage caused by glycation or glycoxidation reactions. Thus, the active principle according to the invention will enable combating the aesthetic damage caused to the skin and/or the hair by free radicals.

Also, the active principle may be used advantageously in a cosmetic composition to protect the skin and the appendages against all types of external aggressions. The expression “external aggressions” is understood to be aggressions which the environment can produce. By way of example, aggressions may be cited such as pollution, UV rays, or even products of an irritating nature such as surfactants, preservatives, and perfumes. Pollution is also understood to be both “outdoor” pollution due, for example, to Diesel-fuel particles, ozone, or heavy metals, as well as “indoor” pollution, which may be due, in particular, to the emissions of the solvents of paint, glue, or wallpaper (such as toluene, styrene, xylene, or benzaldehyde), or even cigarette smoke.

The active principle according to the invention may be advantageously used in a cosmetic composition or for preparing a pharmaceutical composition, as an active photo-protective principle and, more particularly, as a so-called “secondary” active photo-protective principle. Primary active photo-protective principles are, in effect, differentiated from secondary active photo-protective principles. Primary active photo-protective principles are substances that exercise a physical power: they are capable of absorbing UV rays and releasing them in the form of heat in order to protect the skin. Secondary active photo-protective principles are substances that generally have a biological effect; they are, for example, active principles capable of limiting the damage caused to the DNA and to the membranes by UV rays penetrating into the skin.

The invention also has, as an object, use in a cosmetic composition of an effective quantity of active principle as previously described to prevent damage caused to the skin by exposure to the sun or exposure to ionizing radiation during radiotherapy.

The invention also has, as an object, use in a cosmetic composition of an effective quantity of active principle as previously described to increase the synthesis of intracellular ATP in the cells of the skin.

The invention also has, as an object, use in a cosmetic composition of an effective quantity of active principle as previously described to increase the activity or the synthesis of the enzyme transphenyl transferase and/or coenzyme Q10 in the cells of the skin.

The invention again is related to use in a cosmetic composition of an effective quantity of active principle as described previously to protect the skin from damage caused by free radicals.

The invention consists too of the use of an effective quantity of active principle, as described previously, for making a pharmaceutical composition intended to mitigate a pathology linked to oxidation processes or even certain pathologies of aging.

Finally, the invention again consists of a cosmetic-treatment procedure intended to protect the skin and the appendages from external aggressions and to combat cutaneous aging, characterized by the application onto the skin or the appendages to be treated of a composition containing an effective quantity of active principle according to the invention.

Specific embodiments of this cosmetic-treatment procedure also result from the preceding description. Other advantages and characteristics of the invention will be more apparent upon reading the examples given as illustration and non-restrictive.

### EXAMPLE 1

### Preparation of Active Principle Starting with Amaranth (Amaranthus hypochondriacus)

The active agent is obtained starting from an extract of plants of the species Amaranthus hypochondriacus.

In a first step, 1 kg of hulled amaranth seeds is crushed in a cereal crusher and the meal obtained is delipidated by means of the action of an organic solvent, hexane. After filtration and drying in a vacuum, the powder obtained is placed in suspension in an aqueous alkaline solution (dilution to 1/10) of pH 10, containing 1% polyvinyl polypyrrolidone (Polyclar V ISP). This mixture is kept while stirring for a time sufficient to allow solubilization of the soluble fractions. The extraction temperature is variable (between 4 and 80° C.). Preferentially, the operation is carried out cold. After this extraction phase, the medium is clarified by centrifuging and then filtered on a plate filter. This filtrate, which contains the soluble fractions of the amaranth, is then subjected to protein precipitation under varying ionic strength in a neutral or acidic medium, which enables the soluble glucide components, the lipids, and the nucleic acids to be eliminated. The medium is brought to a pH of 3.5. The supernatant is eliminated and the precipitate is then washed using a solvent such as, for example, ethanol or methanol, and then the solvent is evaporated by drying in a vacuum.

At this stage, about 50 grams of clear, yellow-colored powder of raw proteinic extract are obtained, containing:

Proteins: 75%

Glucides: 20%

Lipids: 5%

The precipitate, rich in proteins is placed back into solution in water or another solvent.

The raw proteinic extract is then subjected to a series of controlled and selective hydrolyses consisting of chemical and enzymatic hydrolyses in the presence of 5% PVPP (Polyclar V) and endopeptidases to cysteine (papayine, ficine). After reaction, the hydrolysate is filtered onto a plate and then onto a sterilizing cartridge (0.2 μm).

A clear-colored hydrolysate is then obtained titering from 15 to 30 g/l of dry extract, which is then diluted such that the composition in compounds of a peptide nature determined by Lowry's method is between 0.1 and 5 g/l, and preferentially between 0.5 and 2 g/l. The physico-chemical analysis of the vegetable hydrolysate, which constitutes the active principle, shows that its pH is between 4 and 7, and preferentially between 5 and 6, the dry extract titers between 1 and 8 g/l, and preferably between 2 and 5 g/l. Its content in compounds of a peptide nature is between 0.1 and 5 g/l, and preferentially between 0.5 and 2 g/l, and its content in sugars is from 0.5 to 2.5 g/1.

### EXAMPLE 2

### Preparation of Active Principle Starting with Amaranth (Amaranthus hvpochondriacus)

A variant of the protocol of example 1 consists of carrying out the same sequence of controlled and selective enzymatic hydrolyses, but in the presence of 0.5% PVPP. A clear-colored hydrolysate is then obtained titering 15 to 30 g/l of dry extract after a sterilizing filtration.

You then proceed to ultrafiltration of the solution onto a Helicon millipore filtration cartridge (1 kD cutoff). The high molecular weights contained in the retentate are isolated, and the filtrate is retained.

The concentration in compounds of a peptide nature determined by Lowry's method to be between 0.1 and 5 g/l, and preferentially between 0.5 and 2 g/l. The physico-chemical analysis of the vegetable hydrolysate, which constitutes the active principle, shows that its pH is between 4 and 7, and preferentially between 5 and 6, the dry extract titers between 1 and 8 g/l, and preferably between 2 and 5 g/l. Its content in compounds of a peptide nature is between 0.1 and 5 g/l, and preferentially between 0.5 and 2 g/l, and its content in sugars is from 0.5 to 2.5 g/1.

Another variant consists of carrying out a purification of the active principle obtained according to example 1 or 2, by ion-exchange chromatography, on a gel TSK column (TsosHaas) with a pH 7 phosphate buffer.

### EXAMPLE 3

### Disclosure of the activator effect of the active principle according to example 1 on the synthesis of intracellular ATP

The aim of this study was to determine the effect of the active principle on the synthesis of ATP.

Protocol

This study was conducted using an ATP Bioluminescence Assay Kit HS II (Roche Applied Science). Dermal fibroblasts were treated with a 1% solution of the active principle according to example 1 for a period of 1 to 3 hours. At the end of the incubation time, the tubes were emptied of their medium and rinsed with 2 ml of cold PBS before adding 250 μl of a lysing buffer provided in the kit. The cells of each tube were then scraped out and collected in 14-ml tubes. Each tube was rinsed with 2×500 μl of cold PBS and the whole was collected again in the respective tubes. Starting with these samples, a dilution to 1/12000th was achieved in cold PBS before each reading. ATP measurement was performed on these samples: 50 μl of this dilution was placed in a Luma cuvette and 50 μl of luminol was added. After 10 seconds, the luminescence reading was begun. The values were standardized relative to the quantity of proteins for each sample. The measurements were made using the Biocounter M2010A Luma®/3M equipment.

Results

The ATP measurements show that there is an increase of 17% in the amount of intracellular ATP after one hour and 67% after 3 hours of culture in the cells treated with the active principle, compared to the untreated cells.

Conclusion

The active principle strongly activates the energy level of synthesis of intracellular ATP in cutaneous cells.

### EXAMPLE 4

### Evaluation of the Protective Effect of the Active Principle According to Example 1 with Respect to Oxidative Damage

The aim of this study was to determine the protective effect of the active principle according to example 1 with respect to dermal fibroblasts subjected to an oxidative stress caused by UVB rays or hydrogen peroxide (H₂O₂). In order to evaluate the oxidative damage undergone by the cells, measurements were made of protein carbonylation.

The carbonylation of proteins results from the oxidative cleaving of proteins or from an oxidation of the residues of arginine, lysine, proline, or threonine. Protein carbonylation measurement was done using the (enzyme immunoassay (EIA) technique Protocol

Fibroblasts in culture had been placed in the presence of 1% active principle 72 hours before, during, and after the oxidative stress (UVB irradiation at 50 mJ/cm² or treatment with 2 mM of H₂O₂). Controls untreated and not subjected to the oxidative stress were achieved.

The carbonylation measurement consisted of using DNP (dinitrophenyl), which has the property of being specifically fixed on the carbonyl groups of proteins. Fixed DNP is then measured by an ELISA method, thank to an anti-DNP antibody coupled to a peroxydase. An oxided bovine serum albumin (BSA) scale (which recognizes the concentration in carbonyl groups) is used for standardization.

Results

The results obtained show a reduction of 30% in protein carbonylation when the cells were treated with the active principle according to example 1, compared with untreated cells.

More particularly, a reduction of 20% is observed in the carbonylation when the cells treated with the active principle according to example 1 are subjected to UVB irradiation or to an oxidative stress with H₂O₂, compared with cells irradiated or stressed but not treated with the active principle.

Conclusions

The active principle according to example 1 effectively protects the cutaneous cells against oxidative damage caused by UVB rays or hydrogen peroxide.

### EXAMPLE 5

### Evaluation of the Protective Effect of the Active Principle According to Example 1 with Respect to a Stress Induced by Glycation

The aim of this study was to determine the protective effect of the active principle according to example 1 with respect to an ex vivo epidermal culture subjected to a stress by a glycant agent.

Protocol

Biopsies of human skin were kept in an ex vivo culture, treated with a 1% solution 24 hours before and then 24 hours after being placed in the presence of a glycant agent (5 or 10 mM methyl glyoxal). Slices and histological hematoxyline-eosine (H&E) stainings enabled the quality of the cutaneous structures to be evaluated.

Results

Observation shows a clear reduction of the signs of cellular stress and better preservation of cutaneous structures in the skin biopsies treated with the active principle according to example 1, compared with untreated skin biopsies.

Conclusions

The active principle according to example 1 protects the skin from a stress induced by glycation.

### EXAMPLE 6

### Preparation of Compositions

1. Sunscreen cream

| | | |
|---|---|---|
| | International Nomenclature | |
| | of Cosmetic Ingredients | |
| Trade name | (INCI) names | % W/W |

PHASE A

| | | |
|---|---|---|
| Demineralized water | Aqua (water) | In sufficient |
| | | quantity |
| Pemulen ® TR-1 | Acrylates/C10-30 alkyl | 0.40 |
| | acrylate cross-polymer | |
| Glycerine | Glycerin | 3.00 |
| Nipastat ® Sodium | Sodium methylparaben (and) | 0.15 |
| | sodium ethylparaben (and) | |
| | sodium butylparaben (and) | |
| | sodium propylparaben (and) | |
| | sodium isobutylparaben | |

PHASE B

| | | |
|---|---|---|
| Parsol ® MCX | Ethylhexyl methoxycinnamate | 7.50 |
| Eusolex ® 4360 | Benzophenone-3 | 3.00 |
| Parsol ® 1789 | Butyl methoxydibenzoyl- | 2.00 |
| | methane | |
| Myritol ® 318 | Caprylic/capric triglyceride | 4.00 |
| Emulgade ® SEV | Hydrogenated palm glycerides | 5.00 |
| | (and) ceteareth-20 (and) | |
| | ceteareth-12 (and) cetearyl | |
| | alcohol | |
| Propylparaben | Propylparaben | 0.15 |
| Nacol ® 16-98 | Cetyl alcohol | 1.00 |

PHASE C

| | | |
|---|---|---|
| TEA | Triethanolamine | 0.20 |

PHASE D

| | | |
|---|---|---|
| Active principle | | 3 |
| acc. to ex. 1 | | |
| Perfume | Perfume (fragrance) | In sufficient |
| | | quantity |
| Colorant | | In sufficient |
| | | quantity |

The constituents of phase A and of phase B are heated separately to a temperature between 70° C. and 75° C. Phase B is emulsified in phase A while stirring. Phase C is added, at 45° C., while increasing the stirring. Phase D is then added when the temperature was below 40° C. Cooling is continued down to 25° C. with brisk stirring.

2. After-Sun Milk

| | | |
|---|---|---|
| Trade name | INCI names | % W/W |

PHASE A

| | | |
|---|---|---|
| Montanov ™ L | C14-22 alcohols (and) C12- | 3.00 |
| | 20 alkyl glucoside | |
| Waglinol 2559 | Cetearyl isononanoate | 4.00 |
| Tegosoft ® TN | C12-15 alkyl benzoate | 3.00 |
| Apricot kernel oil | Prunus armeniaca (apricot) | 2.00 |
| | kernel oil | |
| Avocado oil | Persea gratissima (avocado) | 1.00 |
| | oil | |
| Abil ® 350 | Dimethicone | 1.00 |

PHASE B

| | | |
|---|---|---|
| Demineralized water | Aqua (water) | In sufficient |
| | | quantity |

PHASE C

| | | |
|---|---|---|
| Simulgel ™ EG | Sodium acrylate/acryloyl- | 0.4 |
| | dimethyl taurate copolymer | |
| | (and) isohexadecane (and) | |
| | polysorbate 80 copolymer | |
| | (and) polysorbate 80 | |

PHASE D

| | | |
|---|---|---|
| Phenonip ® | Phenoxyethanol (and) | 0.30 |
| | methylparaben (and) | |
| | ethylparaben (and) | |
| | butylparaben (and) | |
| | propylparaben (and) | |
| | isobutylparaben | |
| | ethylparaben and | |
| | propylparaben and | |
| | butylparaben | |
| Germall ® 115 | Imidazolidinyl urea | 0.20 |

PHASE E

| | |
|---|---|
| Active principle | 0.1 |
| acc. to ex. 1 | |

Prepare phase A while stirring. Incorporate the xanthan gum gradually, with dispersant stirring. Phases C and D will be incorporated once the gel has set. Phase E, prepared in advance to the point of perfect DHA dissolution, will then be added. Adjust the pH, if necessary, to 4-4.5. Color and perfume.

3. Anti-Aging Cream

| | | |
|---|---|---|
| Trade name | INCI names | % W/W |

PHASE A

| | | |
|---|---|---|
| Montanov ™ 68 | Cetearyl alcohol (and) | 6.00 |
| | cetearyl glucoside | |
| Squalane | Squalane | 3.00 |
| Cetiol ® SB 45 | Butyrospermum parkii (Shea | 2.00 |
| | butter) | |
| Waglinol 250 | Cetearyl ethylhexanoate | 3.00 |
| Amerchol L-101 | Mineral oil (and) lanolin | 2.00 |
| | alcohol | |
| Abil ® 350 | Dimethicone | 1.50 |
| BHT | Butylhydroxytoluene | 0.01 |
| Coenzyme Q10 | Ubiquinone | 0.10 |

PHASE B

| | | |
|---|---|---|
| Avocado oil | Persea gratissima (avocado) | 1.25 |
| | oil | |
| Phenonip ® | Phenoxyethanol (and) | 0.75 |
| | methylparaben (and)\ | |
| | ethylparaben (and) | |
| | butylparaben (and) | |
| | propylparaben (and) | |
| | isobutylparaben | |

PHASE C

| | | |
|---|---|---|
| Demineralized water | Aqua (water) | In sufficient |
| | | quantity |
| Butylene glycol | Butylene glycol | 2.00 |
| Glucam ™ E10 | Methyl gluceth-10 | 1.00 |
| Allantoin | Allantoin | 0.15 |
| Carbopol ® Ultrez 10 | Carbomer | 0.20 |

PHASE D

| | | |
|---|---|---|
| TEA | Triethanolamine | 0.18 |

PHASE E

| | | |
|---|---|---|
| Active principle | | 0.5 |
| acc. to ex. 1 | | |
| GP4G ® | Water (and) Artemia extract | 1.50 |
| Collaxyl ® | Water (and) butylene glycol | 3.00 |
| | (and) hexapeptide-9 | |

PHASE F

| | | |
|---|---|---|
| Perfume | Perfume (fragrance) | In sufficient |
| | | quantity |
| Colorant | | In sufficient |
| | | quantity |

Prepare and melt phase A at 65-75° C. Heat phase C to 65-70° C. Phase B is added to phase A just before emulsifying A in B. At about 45° C., the carbomer is neutralized by the addition of phase D. Phase E is then added with light stiffing, and cooling is continued to 25° C. Phase F is then added, if desired.

4. Daytime Protection Cream

| | | |
|---|---|---|
| Trade name | INCI names | % W/W |

PHASE A

| | | |
|---|---|---|
| Emulium delta ® | Cetyl alcohol (and) glyceryl | 4.00 |
| | stearate (and) PEG-75 | |
| | stearate (and) ceteth-20 | |
| | (and) steareth-20 | |
| Lanette O | Cetearyl alcohol | 1.50 |
| DC 200 Fluid ®/100 cs | Dimethicone | 1.00 |
| DUB 810C | Coco caprylate/caprate | 1.00 |
| DPPG | Propylene glycol | 3.00 |
| | dipelargonate | |
| DUB DPHCC | Dipentaerythrityl | 1.50 |
| | hexacaprylate/hexacaprate | |
| Cegesoft ® PS 6 | Vegetable oil | 1.00 |
| Vitamin E | Tocopherol | 0.30 |
| Phenonip | Phenoxyethanol (and) | 0.70 |
| | methylparaben (and) | |
| | ethylparaben (and) | |
| | butylparaben (and) | |
| | propylparaben (and) | |
| | isobutylparaben | |

PHASE B

| | | |
|---|---|---|
| Demineralized water | Aqua | In sufficient |
| | | quantity, 100 |
| Glycerine | Glycerin | 2.00 |
| Carbopol ® ETD 2020 | Acrylates/C10-30 alkyl | 0.15 |
| | acrylate cross-polymer | |
| Keltrol CG-BT | Xanthan gum | 0.30 |

PHASE C

| | | |
|---|---|---|
| Sodium hydroxide (10% | Sodium hydroxide | 0.30 |
| solution) | | |

PHASE D

| | | |
|---|---|---|
| Demineralized water | Aqua | 5.00 |
| Stay-C ® 50 | Sodium ascorbyl phosphate | 0.50 |

PHASE E

| | | |
|---|---|---|
| Butylene glycol | Butylene glycol | 2.00 |
| Dekaben CP | Chlorphenesin | 0.20 |

PHASE F

| | | |
|---|---|---|
| GP4G ® | Water (and) Artemia extract | 1.00 |
| Active principle | | 5 |
| acc. to ex. 1 | | |

Prepare phase A and heat to 75° C. while stiffing. Prepare phase B while dispersing the Carbopol® and then the xanthan gum while stirring. Let rest. Heat to 75° C. At temperature, emulsify A in B with rotor-stator stirring. Neutralize with phase C while stirring rapidly. After cooling to 40° C., add phase D and then phase E. Cooling is continued with light stirring and phase F is added.

1-14. (canceled)

15. A method of increasing cellular energy and protecting skin from oxidative damage, comprising adminstrating an effective amount of a peptide active principle, wherein,
said active principle is in a composition, and
said active principle originates from the hydrolysis of proteins from amaranth of the species Amaranthus hypochondriachus.

16. The method according to claim 15, wherein said active principle contains between 0.1 and 5 g/l of compounds of a peptide nature.

17. The method, according to claim 16, wherein said active principle contains between 0.5 and 2 g/l of compounds of a peptide nature.

18. The method according to claim 15, wherein said active principle is used in a quantity representing 0.0001 and 20% of the total weight of the composition.

19. The method according to claim 18, wherein said active principle is used in a quantity representing 0.05 and 5% of the total weight of the composition.

20. The method according to claim 15, wherein said active principle is solubilized in advance in a solvent selected from the group consisting of water, glycerol, ethanol, propylene glycol, butylene glycol, dipropylene glycol, ethoxylated or propoxylated diglycols, cyclic polyols, vaseline, a vegetable oil, and mixtures thereof.

21. The method according to claim 15, said composition is in a form adapted to topical application.

22. The method according to claim 15, wherein said composition further comprises at least one other active agent promoting the action of said active principle.

23. The method according to claim 22, wherein said other active principle is selected from the group consisting of principles having an antioxidant action, active principles stimulating the synthesis of dermal macromolecules, and active principles stimulating energy metabolism.

24. A compound consisting of an active peptide principle, wherein said active principle originates from the hydrolysis of proteins from amaranth of the species Amaranthus hypochondriacus.

25. A method of protecting skin and appendages against external aggression comprising administering to a subject in need thereof an effective amount of the compound accordion to claim 24 in a cosmetic composition to protect the skin and the appendages against external aggression.

26. A method of increasing at least one of the synthesis of intracellular ATP, the activity or synthesis of the enzyme transphenyl transferase, and coenzyme Q10 skin cells comprising administering to a subject in need thereof an effective amount of compound according to claim 24 in a cosmetic composition.

27. A method of prevention or treating skin damage and appendage damage caused by UV radiation, comprising administering to a subject in need thereof an effective amount of compound according to claim 24 in a cosmetic composition.

28. A method of protecting skin and appendages against oxidative damage comprising administering to a subject in need thereof an effective amount of compound according to claim 24 in a cosmetic composition.

29. A method of preventing or treating the cutaneous signs of aging and/or photo-aging, comprising administering to to a subject in need thereof an effective effective amount of compound according to claim 24 in a cosmetic composition.

30. A method of preventing or combating pathologies associated with processes of oxidation, comprising administering to a subject in need thereof an effective effective amount of compound according to claim 24 in a pharmaceutical composition.

31. A method of cosmetic treatment comprising topically administering to a subject in need thereof an effective amount of compound according to claim 24 onto the skin or the appendages to be treated.
